(19) United States
(12) Patent Application Publication
(10) Pub. No.: US 2024/0337737 A1
(43) Pub. Date: Oct. 10, 2024

(54) SYSTEM AND METHODS FOR TRANSMISSION OF NON-DIFFRACTING ACOUSTIC BEAMS

(71) Applicants: GE Precision Healthcare LLC, Wauwatosa, WI (US); The General Hospital Corporation, Boston, MA (US)

(72) Inventors: Yuyang Gu, Boston, MA (US); Anthony E. Samir, Newton, MA (US); Kai Thomenius, Clifton Park, NY (US); Viksit Kumar, Ashburn, VA (US); Michael J. Washburn, Wauwatosa, WI (US); Michael H. Wang, Wauwatosa, WI (US); Rimon Tadross, Wauwatosa, WI (US)

(21) Appl. No.: 18/298,212

(22) Filed: Apr. 10, 2023

(51) Int. Cl. G01S 7/52; A61B 8/00; A61B 8/08; G01S 15/89

(52) U.S. Cl. CPC G01S 7/52022; A61B 8/485; A61B 8/54; G01S 15/8915

(57) ABSTRACT

Systems and methods for transmitting non-diffracting acoustic beams are presented herein. In one example, a method for transmitting a non-diffracting acoustic beam with an ultrasound transducer that includes a plurality of transducer elements includes determining a transmit delay function and a transmit apodization function for the ultrasound transducer based on a target axial pressure profile for the acoustic beam for a given configuration of the ultrasound transducer and controlling the ultrasound transducer to transmit the acoustic beam by sending electrical signals to the plurality of transducer elements based on the transmit delay function and the transmit apodization function.

### TECHNICAL FIELD

Embodiments of the subject matter disclosed herein relate to ultrasound imaging, and more particularly, to transmission of non-diffracting acoustic beams for use in ultrasound imaging.

### BACKGROUND

Medical ultrasound is an imaging modality that employs ultrasound waves to probe the internal structures of a body of a patient and produce a corresponding image. For example, an ultrasound probe comprising a plurality of transducer elements emits ultrasonic pulses which reflect or echo, refract, or are absorbed by structures in the body. The ultrasound probe then receives reflected echoes, which are processed into an image. Ultrasound images of the internal structures may be saved for later analysis by a clinician to aid in diagnosis and/or displayed on a display device in real time or near real time.

### SUMMARY

In one embodiment, a method for transmitting a non-diffracting acoustic beam with an ultrasound transducer that includes a plurality of transducer elements includes determining a transmit delay function and a transmit apodization function for the ultrasound transducer based on a target axial pressure profile for the acoustic beam for a given configuration of the ultrasound transducer and controlling the ultrasound transducer to transmit the acoustic beam by sending electrical signals to the plurality of transducer elements based on the transmit delay function and the transmit apodization function.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 shows a block diagram of an ultrasound system, according to an embodiment;

FIG. 2 is a schematic diagram illustrating a process for producing a non-diffracting acoustic beam from superposed acoustic beam components, according to an embodiment;

FIG. 3 shows example source amplitude and phase of an acoustic source used to produce a non-diffracting acoustic beam, according to an embodiment;

FIG. 4 shows ortho-slices of an acoustic field of various example non-diffracting acoustic beams;

FIG. 5 shows example pressure and phase multiplexing assigned to an acoustic transducer via apodization and delay functions, according to an embodiment;

FIG. 6 shows an acoustic field of an example non-diffracting acoustic beam along with simulated acoustic pressure distributions of the non-diffracting acoustic beam;

FIG. 7 shows example shear waves that may be generated with non-diffracting acoustic beams according to embodiments of the disclosure;

FIG. 8 is a flow chart illustrating a method for performing ultrasound imaging using non-diffracting acoustic beams, according to an embodiment of the disclosure;

FIG. 9 is a flow chart illustrating a method for determining apodization and delay functions for generating a non-diffracting acoustic beam with an ultrasound probe, according to an embodiment of the disclosure;

FIG. 10 shows simulated acoustic pressure and corresponding plots of pressure distribution of non-diffracting acoustic beams with and without attenuation and compensation;

FIG. 11 schematically shows an example process for performing phase-only beamshaping for forming a non-diffracting acoustic beam;

FIG. 12 shows example elastograms generated with non-diffracting acoustic beams shaped using phase-only modulation;

FIG. 13 shows various example non-diffracting acoustic beams that differ in beam length and beam depth;

FIG. 14 shows an example non-diffracting acoustic beam profile and different apodization and delay functions that may be applied to form the non-diffracting acoustic beam using different transducer configurations;

FIG. 15 shows multiple example non-diffracting acoustic beams formed with the same transducer;

FIG. 16 shows an example non-diffracting acoustic beam formed from a split aperture; and

FIG. 17 shows example target axial pressure profiles for generating non-diffracting acoustic beams.

### DETAILED DESCRIPTION

Ultrasound images acquired during a medical ultrasound exam may be used to diagnose a patient condition, which may include one or more clinicians analyzing the ultrasound images for abnormalities, measuring certain anatomical features imaged in the ultrasound images, and so forth. Shear wave imaging (e.g., elastography imaging), which is a mechanism for non-invasively measuring tissue elasticity (e.g., stiffness), may be employed in certain clinical settings, such as imaging a liver to determine stiffness in the context of cirrhosis diagnosis. Acoustic (e.g., ultrasound) beam shaping and degrees of freedom for acoustic beam shaping affects shear wave imaging, acoustic manipulation, and stimulation during imaging.

A conventional ultrasound beam employed for shear wave imaging, such as acoustic radiation-force impulse (ARFI) shear wave imaging, is a focused beam. However, the resulting shear wave image has a limited extent of the focal zone. While increasing an F-number (ratio of focal length to diameter of aperture) may extend a depth of the field, doing so also decreases acoustic intensity of the beam which may result in insufficient intensities at near and far fields.

Thus, various acoustic-wave modulation methods have been introduced to achieve a more comprehensive and controllable acoustic field during imaging. However, the ability to fully control the acoustic profile over its propagation path (e.g., longitudinal path) is difficult. Propagation-invariant (e.g., non-diffracting) beams have been used to increase control of the acoustic profile by sustaining longitudinal pressure distribution invariance over an extended range during image acquisition. Types of propagation-invariant beams include Bessel beams, Airy beams, Mathieu beams, and Weber beams, each including features aimed at different applications. Bessel beams and Airy beams, for instance, are often aimed in acoustic applications. Current acoustic propagation-invariant beams have longitudinal pressure distributions that are fixed, which hinders practical usefulness. For example, while Bessel-apodized beams may cover the near field with an extended depth-of-field (DOF), the beam profile being fixed results in insufficient amplitude in the far field. As a result, conventional non-diffracting beams may be insufficient for practical applications such as liver fibrosis imaging.

The systems and methods described herein provide for generation of a flexible non-diffracting acoustic beam with an extended focal-zone and DOF that, when used as a push beam to generate a shear wave for ARFI shear-wave imaging, increases the region of high intensity that provides an extended effective imaging area with a broader accurate shear-wave speed map and improved delineation of inclusions. The acoustic beams herein described, hereafter referred to as non-diffracting acoustic beams, may realize arbitrary longitudinal pressure distribution and may be generated with a linear array transducer via multiplexing of multiple acoustic beam components. Multiple variations are disclosed for medical ultrasound, including attenuation compensation and beam shaping using only phase delay. The process for generating the non-diffracting acoustic beams described herein may be referred to as acoustic diffraction-resistant adaptive profile technology (ADAPT) and thus the non-diffracting acoustic beams may also be referred to as ADAPT-based beams or non-diffracting acoustic beams formed using ADAPT.

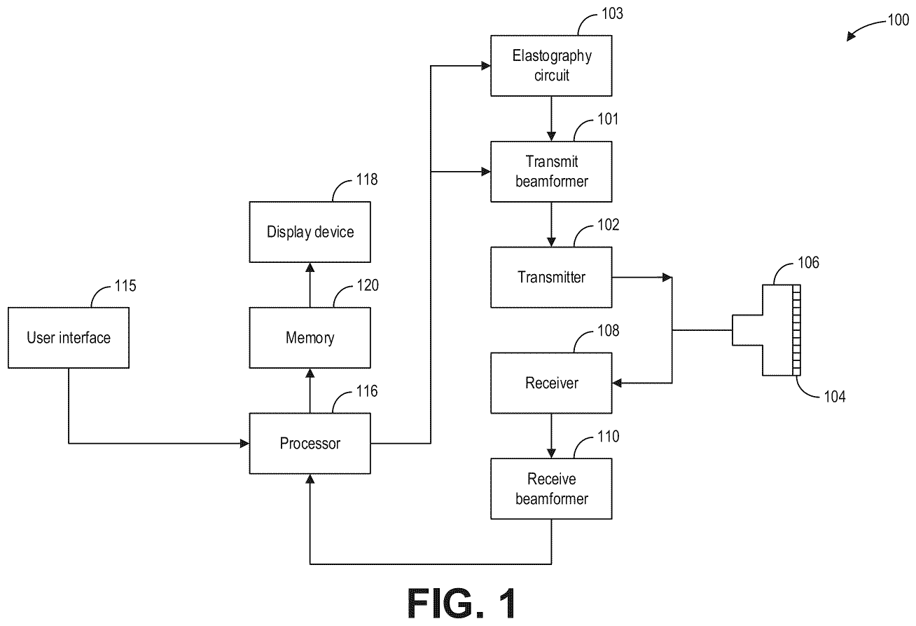

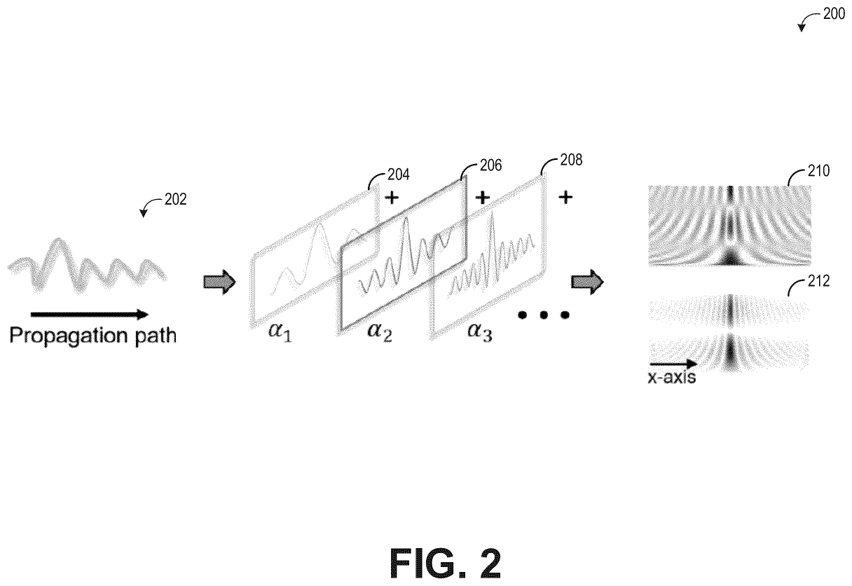

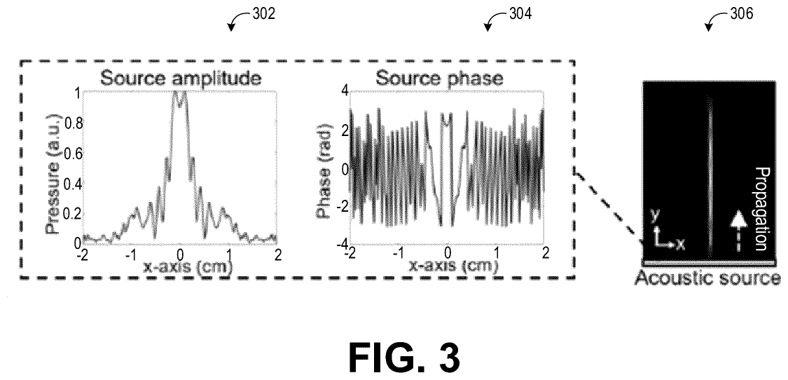

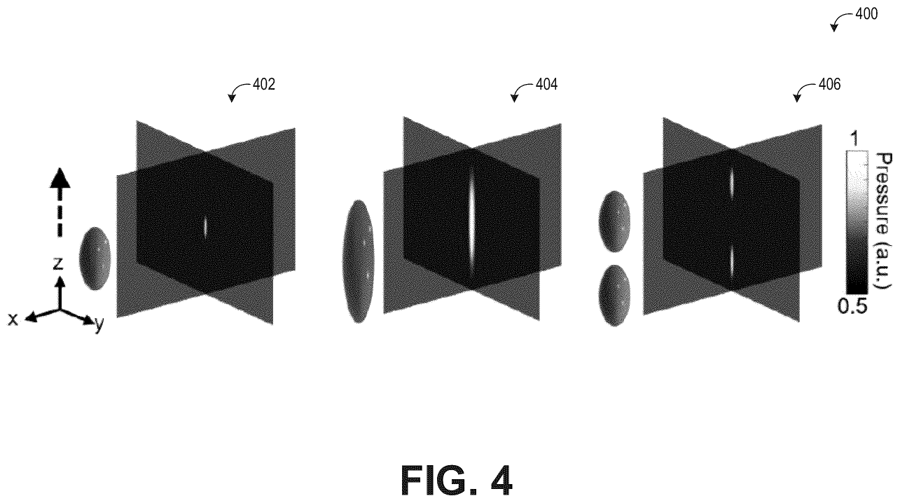

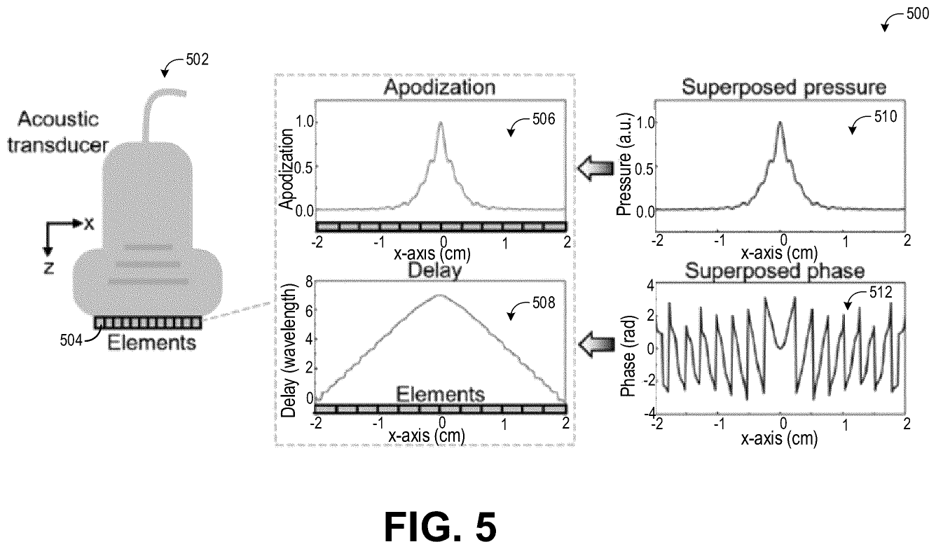

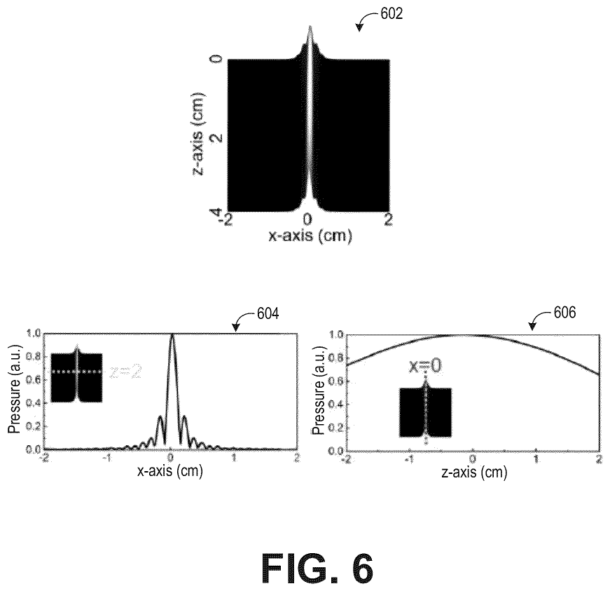

An ultrasound imaging system, such as the ultrasound imaging system of FIG. 1, may be used to generate non-diffracting acoustic beams to obtain shear wave (e.g., elastographic) images, for example. The acoustic beams disclosed herein may be formed from multiple beams (also referred to as acoustic beam components) with different wavenumbers that are superposed to form a single non-diffracting acoustic beam, as shown in FIG. 2, with a pressure and phase distribution of the ultrasound transducer, as shown in FIG. 3. Different non-diffracting acoustic beams of varying depth, length, and number may be produced with the same transducer or different transducers, as shown in FIG. 4 and FIGS. 13-16. The superposed acoustic beam components may define an apodization function and a delay function that may be applied to a transducer to form the non-diffracting acoustic beam, as shown in FIG. 5, having a given acoustic pressure distribution along multiple axes, as shown in FIG. 6.

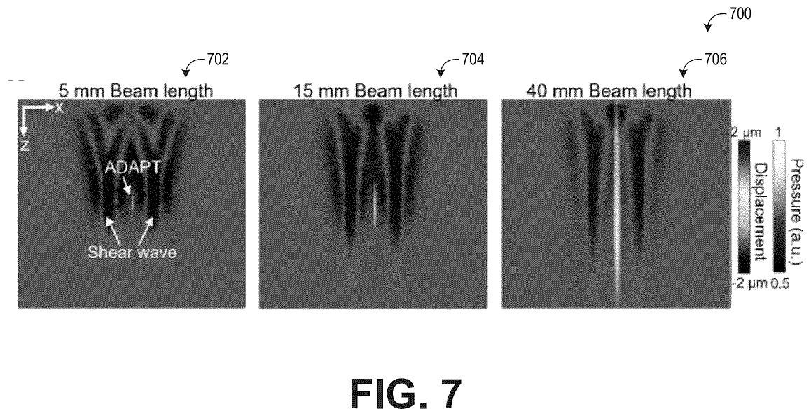

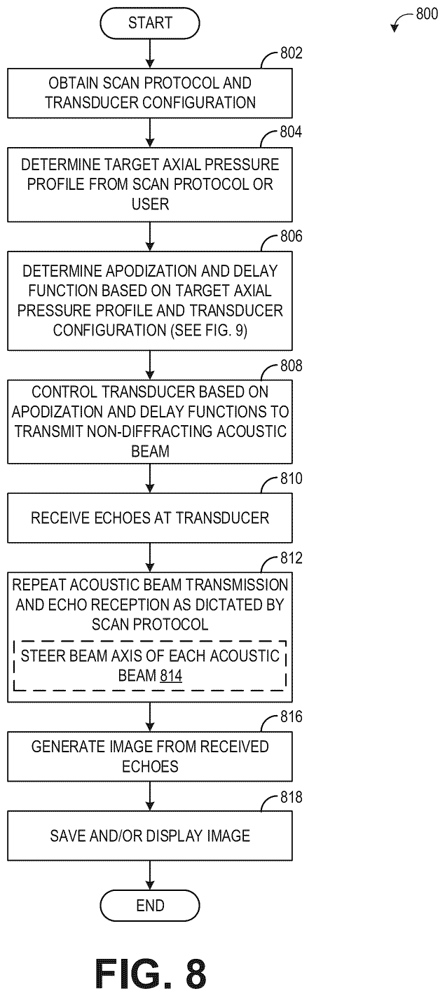

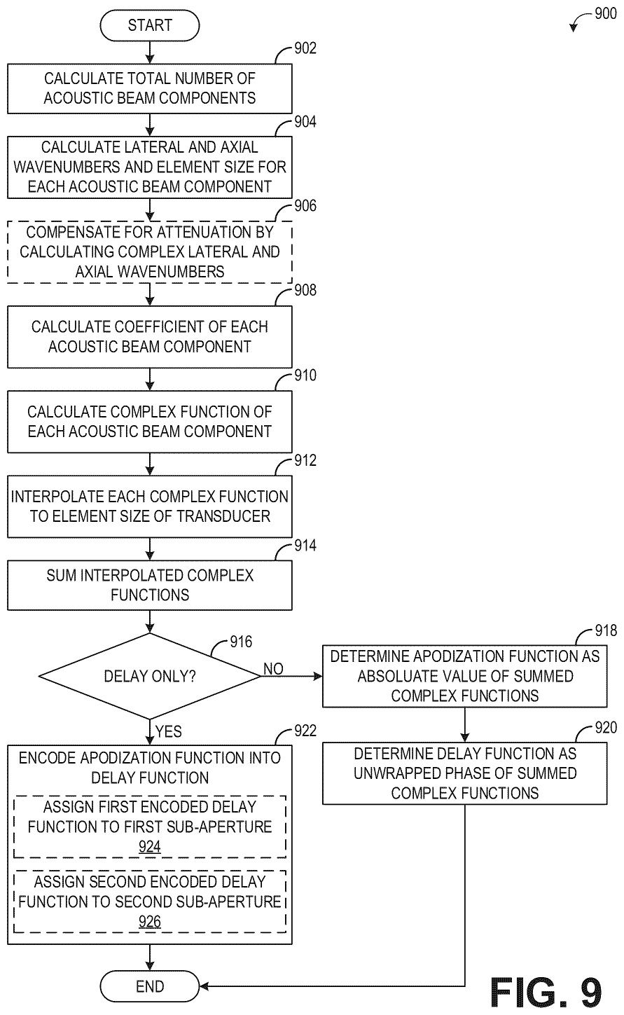

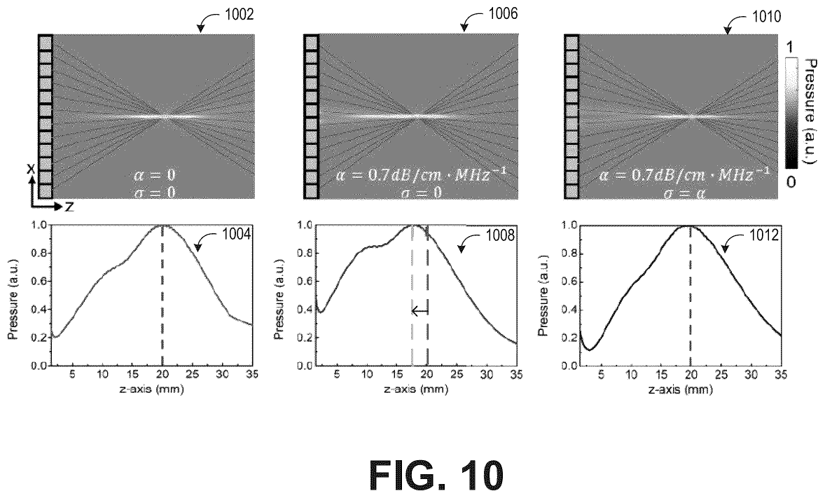

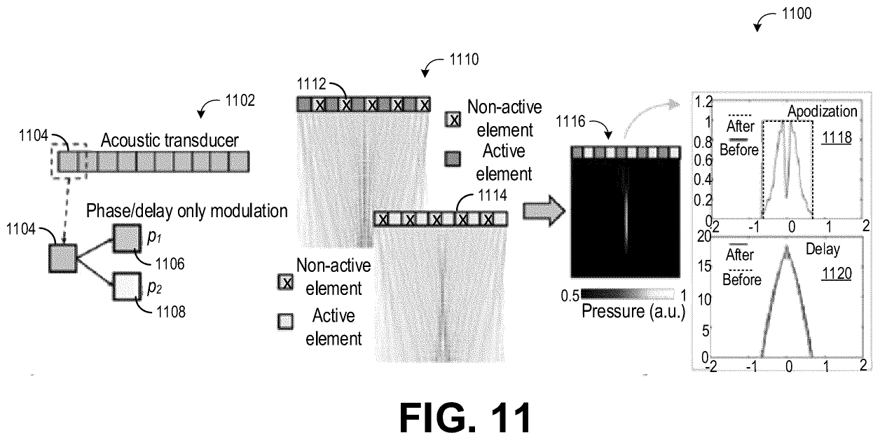

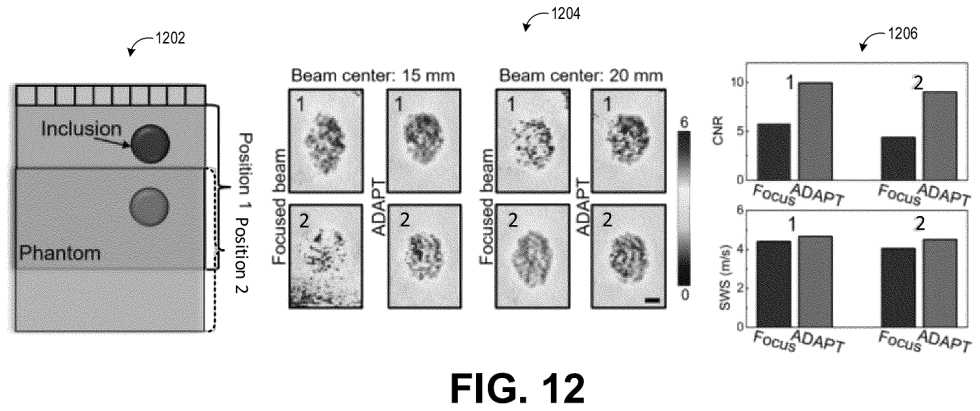

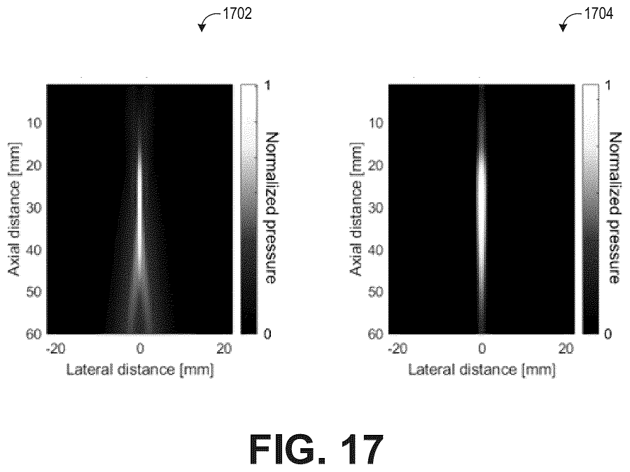

As mentioned previously, non-diffracting acoustic beams may be used as push beams that form shear waves in a tissue of interest, which is shown in FIG. 7, which may be used in shear wave imaging (also referred to as elastography). To image a subject with non-diffracting acoustic beams, an ultrasound system (such as the ultrasound system of FIG. 1), may be controlled according to the method of FIG. 8, whereby an apodization and a delay function are determined to form a non-diffracting acoustic beam with a target axial pressure profile, according to the method of FIG. 9. Example target axial pressure profiles are shown in FIG. 17. During imaging, differences in attenuation of various tissues may be compensated, which may result in decreased distortion of the axial pressure profile and location of the acoustic beam, as shown in FIG. 10. In some examples, rather than separately applying both an apodization function and a delay function to form a non-diffracting acoustic beam, the apodization function may be encoded in the delay function and the transducer controlled according to only the encoded delay function, as shown schematically in FIG. 11, which may be applied during shear-wave imaging to produce elastograms, as shown in FIG. 12.

Referring to FIG. 1, a schematic diagram of an ultrasound imaging system 100 in accordance with an embodiment of the disclosure is shown. The ultrasound imaging system 100 includes a transmit beamformer 101 and a transmitter 102 that drives elements (e.g., transducer elements) 104 within a transducer array, herein referred to as probe 106, to emit pulsed ultrasonic signals (referred to herein as transmit pulses) into a body (not shown). According to an embodiment, the probe 106 may be a one-dimensional transducer array probe. However, in some embodiments, the probe 106 may be a two-dimensional matrix transducer array probe. As explained further below, the transducer elements 104 may be comprised of a piezoelectric material. When a voltage is applied to a piezoelectric crystal, the crystal physically expands and contracts, emitting an ultrasonic spherical wave. In this way, transducer elements 104 may convert electronic transmit signals into acoustic transmit beams.

After the elements 104 of the probe 106 emit pulsed ultrasonic signals into a body (of a patient), the pulsed ultrasonic signals are back-scattered from structures within an interior of the body, like blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that outputs radio frequency (RF) data. Additionally, transducer element 104 may produce one or more ultrasonic pulses to form one or more transmit beams in accordance with the received echoes.

According to some embodiments, the probe 106 may contain electronic circuitry to do all or part of the transmit beamforming and/or the receive beamforming. For example, all or part of the transmit beamformer 101, the transmitter 102, the receiver 108, and the receive beamformer 110 may be situated within the probe 106. The terms “scan” or “scanning” may also be used in this disclosure to refer to acquiring data through the process of transmitting and receiving ultrasonic signals. The term “data” may be used in this disclosure to refer to either one or more datasets acquired with an ultrasound imaging system. A user interface 115 may be used to control operation of the ultrasound imaging system 100, including to control the input of patient data (e.g., patient medical history), to change a scanning or display parameter, to initiate a particular acquisition mode such as shear wave imaging, and the like. The user interface 115 may include one or more of the following: a rotary element, a mouse, a keyboard, a trackball, hard keys linked to specific actions, soft keys that may be configured to control different functions, and a graphical user interface displayed on a display device 118.

The ultrasound imaging system 100 also includes a processor 116 to control the transmit beamformer 101, the transmitter 102, the receiver 108, and the receive beamformer 110. The processor 116 is in electronic communication (e.g., communicatively connected) with the probe 106. For purposes of this disclosure, the term “electronic communication” may be defined to include both wired and wireless communications. The processor 116 may control the probe 106 to acquire data according to instructions stored on a memory of the processor, and/or memory 120. The processor 116 controls which of the elements 104 are active and the shape of a beam emitted from the probe 106. The processor 116 is also in electronic communication with the display device 118, and the processor 116 may process the data (e.g., ultrasound data) into images for display on the display device 118. The processor 116 may include a central processor (CPU), according to an embodiment. According to other embodiments, the processor 116 may include other electronic components capable of carrying out processing functions, such as a digital signal processor, a field-programmable gate array (FPGA), or a graphic board. According to other embodiments, the processor 116 may include multiple electronic components capable of carrying out processing functions. For example, the processor 116 may include two or more electronic components selected from a list of electronic components including: a central processor, a digital signal processor, a field-programmable gate array, and a graphic board. According to another embodiment, the processor 116 may also include a complex demodulator (not shown) that demodulates the RF data and generates IQ data pairs representative of the echo signals. In another embodiment, the demodulation can be carried out earlier in the processing chain. The processor 116 is adapted to perform one or more processing operations according to a plurality of selectable ultrasound modalities on the data. In one example, the data may be processed in real-time during a scanning session as the echo signals are received by receiver 108 and transmitted to processor 116. For the purposes of this disclosure, the term “real-time” is defined to include a procedure that is performed without any intentional delay. For example, an embodiment may acquire images at a real-time rate of 7-20 frames/sec. The ultrasound imaging system 100 may acquire 2D data of one or more planes at a significantly faster rate. However, it should be understood that the real-time frame-rate may be dependent on the length of time that it takes to acquire each frame of data for display. Accordingly, when acquiring a relatively large amount of data, the real-time frame-rate may be slower. Thus, some embodiments may have real-time frame-rates that are considerably faster than 20 frames/see while other embodiments may have real-time frame-rates slower than 7 frames/sec. The data may be stored temporarily in a buffer (not shown) during a scanning session and processed in less than real-time in a live or off-line operation. Some embodiments of the invention may include multiple processors (not shown) to handle the processing tasks that are handled by processor 116 according to the exemplary embodiment described hereinabove. For example, a first processor may be utilized to demodulate and decimate the RF signal while a second processor may be used to further process the data, for example by augmenting the data, prior to displaying an image. It should be appreciated that other embodiments may use a different arrangement of processors.

The ultrasound imaging system 100 may continuously acquire data at a frame-rate of, for example, 10 Hz to 30 Hz (e.g., 10 to 30 frames per second). Images generated from the data may be refreshed at a similar frame-rate on display device 118. Other embodiments may acquire and display data at different rates. For example, some embodiments may acquire data at a frame-rate of less than 10 Hz or greater than 30 Hz depending on the size of the frame and the intended application. A memory 120 is included for storing processed frames of acquired data. In an exemplary embodiment, the memory 120 is of sufficient capacity to store at least several seconds' worth of frames of ultrasound data. The frames of data are stored in a manner to facilitate retrieval thereof according to its order or time of acquisition. The memory 120 may comprise any known data storage medium.

In various embodiments of the present invention, data may be processed in different mode-related modules by the processor 116 (e.g., B-mode, Color Doppler, M-mode, Color M-mode, spectral Doppler, Elastography, TVI, strain, strain rate, and the like) to form two-dimensional (2D) or three-dimensional (3D) data. For example, one or more modules may generate B-mode, color Doppler, M-mode, color M-mode, spectral Doppler, Elastography, TVI, strain, strain rate, and combinations thereof, and the like. As one example, the one or more modules may process color Doppler data, which may include traditional color flow Doppler, power Doppler, HD flow, and the like. The image lines and/or frames are stored in memory and may include timing information indicating a time at which the image lines and/or frames were stored in memory. The modules may include, for example, a scan conversion module to perform scan conversion operations to convert the acquired images from beam space coordinates to display space coordinates. A video processor module may be provided that reads the acquired images from a memory and displays an image in real time while a procedure (e.g., ultrasound imaging) is being performed on a patient. The video processor module may include a separate image memory, and the ultrasound images may be written to the image memory in order to be read and displayed by display device 118.

The ultrasound imaging system 100 includes an elastography circuit 103 configured to enable shear-wave and/or stain elastography imaging. While in the shear-wave mode, the elastography circuit 103 may control the probe 106 to transmit a push beam to generate a shear wave at a site within a region of interest (ROI) of an imaging subject (e.g., a patient). The elastography circuit 103 may control the probe 106 or, more particularly, the transducer elements 104 to direct a shear-wave generating or pushing pulse(s) toward the predetermined site to generate the shear-wave. Alternatively, the elastography circuit 103 may control another device capable of generating shear-waves and the probe 106 may measure or track the velocity as the shear-wave passes through the ROI. For example, the elastography circuit 103 may control a therapy transducer, a mechanical actuator, or an audio device to generate the shear waves. While the elastography circuit 103 is shown as a separate circuit from memory 120 and processor 116, it is to be appreciated that the elastography circuit 103 may comprise instructions stored in memory 120 that are executable by processor 116 to control the ultrasound probe 106 to generate the push beam for elastography imaging.

While in the strain mode, the elastography circuit 103 may control the probe 106 to generate a mechanical force (e.g., surface vibration, freehand or step quasi-static surface displacement, or the like) or radiation force on the patient or ROI to measure the stiffness or strain of the ROI of the patient. Alternatively, the elastography circuit 103 may control another device capable of generating a mechanical force on the patient or the ROI. For example, a low frequency mechanical vibrator may be applied to the skin surface and the compression motion induced in the underlying tissue, such as on the ROI, is measured by the probe 106.

In various embodiments of the present disclosure, one or more components of ultrasound imaging system 100 may be included in a portable, handheld ultrasound imaging device. For example, display device 118 and user interface 115 may be integrated into an exterior surface of the handheld ultrasound imaging device, which may further contain processor 116 and memory 120. Probe 106 may comprise a handheld probe in electronic communication with the handheld ultrasound imaging device to collect raw ultrasound data. Transmit beamformer 101, transmitter 102, receiver 108, and receive beamformer 110 may be included in the same or different portions of the ultrasound imaging system 100. For example, transmit beamformer 101, transmitter 102, receiver 108, and receive beamformer 110 may be included in the handheld ultrasound imaging device, the probe, and combinations thereof.

After performing a two-dimensional ultrasound scan, a block of data comprising scan lines and their samples is generated. After back-end filters are applied, a process known as scan conversion is performed to transform the two-dimensional data block into a displayable bitmap image with additional scan information such as depths, angles of each scan line, and so on. During scan conversion, an interpolation technique is applied to fill missing holes (i.e., pixels) in the resulting image. These missing pixels occur because each element of the two-dimensional block should typically cover many pixels in the resulting image. For example, in current ultrasound imaging systems, a bicubic interpolation is applied which leverages neighboring elements of the two-dimensional block. As a result, if the two-dimensional block is relatively small in comparison to the size of the bitmap image, the scan-converted image will include areas of poor or low resolution, especially for areas of greater depth.

As mentioned previously, an ultrasound probe (such as the ultrasound probe of FIG. 1) with a transducer comprised of a plurality of transducer elements (e.g., piezoelectric elements) arranged into a linear, curved, and/or two-dimensional array, may be controlled to transmit a non-diffracting acoustic beam with a desired axial pressure profile. The non-diffracting acoustic beam may be shaped to have a desired beam length and beam depth for different shear wave imaging needs, as well as shaped for B-mode images, Doppler images, and the like. Moreover, with the flexible capability of the beam shaping method, the acoustic attenuation in the material can be compensated, leading to a more precise beam profile that can generate acoustic radiation force practically at the desired position.

As used herein, an ultrasound transducer (or simply a transducer) may refer to a plurality of ultrasound transducer elements that are each capable of having a defined pressure and phase. In some examples, the ultrasound transducer may include each transducer element of the ultrasound probe. In other examples, the ultrasound transducer may include less than all transducer elements of the ultrasound probe. In still further examples, the ultrasound probe may include or be segmented into multiple ultrasound transducers to enable simultaneous transmission of multiple non-diffracting acoustic beams. Thus, an ultrasound transducer may be flexibly defined based on specific imaging needs or desired location(s) of the non-diffracting acoustic beam(s).

FIG. 2 shows a schematic of a beam-shaping procedure 200 for forming a non-diffracting beam according to the disclosure. In FIG. 2 and remaining figures, the x-axis is the lateral axis of the transducer, e.g., the axis along which the transducer elements are arranged, the z-axis is the axial axis (e.g., the propagation axis of the non-diffracting acoustic beam), and the y-axis is the elevation axis and is orthogonal to each of the z-axis and the x-axis. An arbitrary axial pressure profile 202 (e.g., acoustic pressure along the z-axis/propagation path of the beam, which is an axial direction relative to the ultrasound transducer) can be expanded as a superposition of various aligned acoustic beam components with corresponding weights. For example, a non-diffracting acoustic beam having the axial pressure profile 202 may be formed from a plurality of acoustic beam components, such as first acoustic beam component 204, a second acoustic beam component 206, a third acoustic beam component 208, etc. Each acoustic beam component may be a Bessel beam and each acoustic beam component may have a different axial acoustic profile. Each acoustic beam component includes a respective weight (e.g., first acoustic beam component 204 includes a first weight α1, second acoustic beam component 206 includes a second weight α2, etc.).

Producing the Bessel beams simultaneously using a single acoustic transducer is challenging. Unlike propagation-invariant laser beams generated using a combination of multiple lenses and photomasks, the acoustic propagation-invariant wave typically has a narrower spatial frequency bandwidth, corresponding to limited modulation capability. Linear-array transducers producing the acoustic propagation-invariant wave have a relatively low frequency bandwidth and a bigger element size than an optical wave. Therefore, a multiplexing technique may be used to generate the non-diffracting acoustic beams with the desired features described herein.

A complex function of each acoustic beam component may be determined, including a real part (e.g., real part 210) and an imaginary part (e.g., imaginary part 212), and the complex functions may be interpolated to the resolution (e.g., element size) of the transducer of the ultrasound probe. The non-diffracting acoustic beam can be generated by multiplexing (e.g., summing) the interpolated complex functions, each weighted according to its respective weight.

Thus, the procedure for generating a non-diffracting acoustic beam with a desired axial pressure profile includes separating a single acoustic beam into multiple beams with different wavenumbers. The individual beams can be flexibly defined. However, the superposition of these individual beams is constrained by the desired axial pressure profile. The final acoustic beam pressure (p(x,y,z,t)) is the superposition of the pressure of the acoustic beam components, such that p(x,y,z,t)=Σpₙ(x,y,z,t). A single non-diffracting acoustic beam with an arbitrary pressure profile comprises 0th-order Bessel beams with different wavenumbers and weights. Because a normalized Bessel beam is equal to unity at the origin (i.e., J₀(0)=1), the axial acoustic pressure at x=0 equals the sum of the pressures contributed by all constituent acoustic beams and is independent of their phase distribution. The desired acoustic beam profile (e.g., an axial pressure profile) may be expanded to multiple 0th-order Bessel beams by utilizing a Fourier series expansion. The calculated coefficients of the Fourier series are assigned as the respective weight for each Bessel beam/acoustic beam component. As used herein, a Bessel beam is a wave with an amplitude described by a Bessel function of the first kind and that is non-diffractive (at least over a certain distance). A 0^{th}-order Bessel beam is a Bessel beam that has an amplitude maximum at the origin (e.g., at the ultrasound transducer).

The acoustic beam components may be mapped to the resolution of the transducer. The beam with the highest transverse spatial frequency is aligned to the element size that utilizes the total spatial bandwidth of the transducer (K_{x,max}=2π/Δx, where Δx is the array pitch) to produce multiple acoustic beams simultaneously with different wavenumbers. Other beams with smaller spatial frequencies (e.g., larger element sizes) treat multiple combined elements as sub-elements of a larger effective “element” with the same pressure and phase assigned to each sub-element. Thus, the general procedure of the multiplexing first determines each 0th-order Bessel beam (Bessel functions with coefficients equal to the transverse wavenumbers) corresponding to the largest available spatial frequency. Multiple Bessel functions are calculated with different element numbers (e.g., the number of coordinates in the x-axis). Then, spline interpolation is performed to interpolate each Bessel function to the locations of the transducer elements. The final step is weighting the Bessel function for each component and summing the set of functions. This method enables the multiplexing of a set of Bessel beams and generates a complex value that can be assigned to each transducer element. An advantage of this multiplexing method is that the element dimension does not have to be integer multiples of the element size since the interpolation process ensures the proper alignment value for each element. Therefore, more Bessel beams with finer wavenumber increments can be selected to obtain a better-specified non-diffracting acoustic beam.

Once the complex value of each element eₘ=Aₘe^{iϕ} is calculated, the distribution of the amplitude and phase for the elements of the linear-array transducer may be obtained. The final pressure magnitude and phase can be transferred to the apodization and delay function of the linear array to generate the non-diffracting beam, where the apodization function is the normalized pressure distribution, and the delay function is the unwrapped phase distribution. The ultrasound probe may be controlled to transmit the non-diffracting acoustic beam by controlling each transducer element according to a delay value and an apodization value based on the delay function and apodization function. For example, an electrical signal may be sent to each transducer element that is delayed by the delay value for that transducer element and has a voltage based on the apodization value for that transducer element. FIG. 3 shows the amplitude and phase distribution of each pixel on the acoustic source that results from coherent summing of the set of weighted Bessel functions. The 2D design can be extended to a 3D non-diffracting acoustic beam by rotating the acoustic source plane along its origin, which gives a 2D distribution of the pressure and phase. Thus, as shown in FIG. 3, the acoustic source (e.g., the ultrasound transducer) may have an amplitude profile 302 and a phase profile 304 that produces a non-diffracting acoustic beam 306. The non-diffracting acoustic beam 306 is represented as the acoustic pressure field, with the lighter color regions indicating increased pressure relative to the darker colored regions and has a pre-defined pressure profile in the first position. A sawtooth-like high-pressure region is apparent as an “isolated” and propagation-invariant effect. Because a set of Bessel beams forms the non-diffracting acoustic beam 306, it has a non-diffraction property and markedly diminishes pressure outside the desired high-pressure region by wave interference.

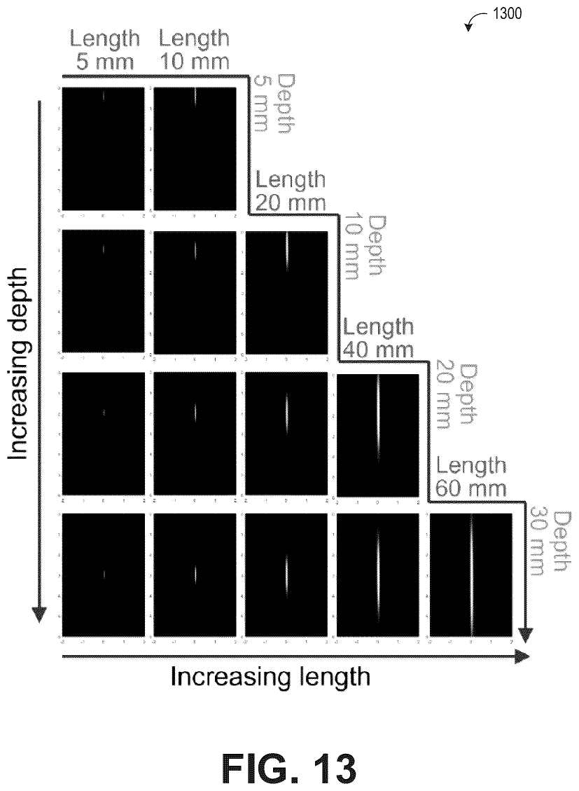

FIG. 4 shows ortho-slices of various non-diffracting acoustic beams 400 that can be created using the ADAPT method disclosed herein. The non-diffracting acoustic beams can be compressed, stretched, and divided into different shapes as desired. For example, FIG. 4 shows a short beam 402, a long beam 404, and a double beam 406. Each beam in FIG. 4 is shown as an acoustic pressure field with slices across the zx-plane and the zy-plane, to illustrate the beams in 3D. Three examples are shown that present different beam profiles formed by the ADAPT method disclosed herein with a high-pressure region flexibly stretched, compressed, or divided. These results illustrate how the ADAPT method described herein can generate non-diffracting acoustic beams at different axial locations with extended high-pressure regions having arbitrary longitudinal pressure profiles. Likewise, FIG. 13 shows a plurality of non-diffracting acoustic beams 1300 formed by the methods disclosed herein, with the non-diffracting acoustic beams increasing in length (which defines the axial/longitudinal start and end points of the beam relative to the transducer) and depth (which defines the axial center point of the beam relative to the transducer).

FIG. 5 schematically shows an example process 500 for generation of a non-diffracting acoustic beam with a single acoustic transducer via beam multiplexing. A schematic of an ultrasound probe 502 is shown, including linear-array transducer 504 comprising multiple transducer elements each with controllable pressure and phase. An apodization function 506 and a delay function 508 may be assigned to the transducer 504. The apodization function 506 may define the apodization value to be applied to each transducer element of the transducer 504, which are indicated along the x-axis of the apodization function 506. The center-most transducer element is assigned the number 0 while elements to the right of 0 are positioned with a positive distance from the center and elements to the left of 0 are positioned with a negative distance from the center. Likewise, the delay function 508 may define the delay value (here shown in wavelength) to be applied to each transducer element of the transducer 504.

FIG. 5 also includes a superposed pressure plot 510 and a superposed phase plot 512, which are the distribution of the amplitude and phase, respectively, for the elements of the transducer 504 determined from the complex value of each element obtained via the multiplexing, which can be assigned to the acoustic transducer as the apodization function 506 and the delay function 508, respectively, where the apodization function is the normalized pressure distribution and the delay function is the unwrapped phase distribution. FIG. 6 shows an acoustic field 602 of an example non-diffracting acoustic beam with a localized pressure distribution along the axial center line, and the transverse pressure distribution 604 (e.g., along the x-axis/horizontal center line) and the longitudinal pressure distribution 606 (e.g., along the z-axis/axial center line) of the non-diffracting acoustic beam, which could span the entire region of interest. The transverse pressure distribution 604 was obtained by measuring the pressure along the x-axis at a distance spaced away from the transducer along the z-axis (herein, a distance of 2 cm; z=2) and the longitudinal pressure distribution 606 was obtained by measuring the pressure along the z-axis at the axial center line (e.g., at x=0).

As mentioned previously, non-diffracting acoustic beams may be used as push beams to generate shear waves in a tissue of interest for shear-wave elastography imaging. An acoustic radiation force is generated proportional to the rate of change of momentum of an acoustic wave propagating in a medium. The force is applied in the direction of wave propagation, and the amplitude of the force per unit volume can be estimated from F=2αI/c, where a is the absorption coefficient of the medium, I is the temporal average acoustic intensity at the given location, and c is the speed of sound in the medium. ARFI produces a push beam that excites a transient laterally propagating shear wave following the acoustic beam profile. The shear-wave speed is directly related to the elasticity properties of the medium, which provides an effective way of evaluating the stiffness of human tissues. While traditional ARFI-enabled shear-wave generation and imaging use a focused beam, the non-diffracting beams disclosed herein generate improved shear waves in soft tissue. FIG. 7 shows non-diffracting acoustic beam-based ARFI push beams with different beam lengths generated inside a tissue-mimicking phantom and corresponding shear waves generated from the push beams. FIG. 7 specifically shows displacement-time profiles for the generated shear waves overlaid with the acoustic fields for the non-diffracting acoustic beams (labeled ADAPT in FIG. 7), for a first non-diffracting acoustic beam 702, a second non-diffracting acoustic beam 704, and a third non-diffracting acoustic beam 706. The centers of three non-diffracting acoustic beams are 20 mm in depth, and the beam lengths are 5 mm, 15 mm, and 40 mm, respectively. A plane-wave compounding method was used to record the in-phase/quadrature (I/Q) data for transient shear-wave motion capture and a Kasai autocorrelation algorithm was applied to calculate the displacement-time profile for the generated shear waves. The non-diffracting acoustic beam acoustic fields are overlaid with the experimentally measured shear-wave profiles (i.e., the displacement map at a particular time frame) in FIG. 7. Note that the shear-wave profile is extended with a longer longitudinal beam length, and the penetration depth is increased, which verifies the expected non-diffracting acoustic beam profile. Thus, the ADAPT process to produce non-diffracting acoustic beams is able to induce the shear wave in an elastic medium through acoustic radiation force. With various beam lengths of non-diffracting acoustic beams, shear waves with different wave profiles may be produced.

FIG. 8 illustrates a method 800 for performing ultrasound imaging using non-diffracting acoustic beams generated according to the ADAPT process. Method 800 is described with regard to the systems and components of FIG. 1, though it should be appreciated that the method 800 may be implemented with other systems and components without departing from the scope of the present disclosure. Method 800 may be carried out according to instructions stored in non-transitory memory of a computing device, such as memory 120, and executed by a processor of the computing device, such as processor 116.

At 802, method 800 includes obtaining a scan protocol and a transducer configuration. The scan protocol may include one or more acquisition modes that the ultrasound probe is to operate under during a scan of an imaging subject (e.g., patient). Example acquisition modes may include B-mode, M-mode, Doppler, shear-wave elastography imaging, and so forth. The scan protocol may further include information relating to the imaging subject (e.g., height, weight) as well as one or more regions of interest to be imaged during the scan of the imaging subject. The scan protocol may be obtained via user input (e.g., a user of the ultrasound system may enter input specifying one or more or each parameter of the scan protocol and/or the user may select a predefined scan protocol from a menu) or obtained automatically based on information of the imaging subject. The acquisition mode that the ultrasound probe is currently operating in may be determined from the scan protocol and/or via user input (e.g., the user may select a desired acquisition mode from a menu or via a control button on the ultrasound probe).

The transducer configuration may specify physical characteristics of the ultrasound transducer of the ultrasound probe, such as a frequency (f) of the ultrasound transducer, a number (n) of transducer elements comprising the ultrasound transducer, a coordinate (x) of each transducer element along the x axis of the ultrasound transducer, and a dimension (dx) of each transducer element (e.g., a width of each element along the x-axis). The transducer configuration may be determined based on the type of ultrasound probe coupled to the ultrasound system, which may be obtained via user input, via the scan protocol, and/or via communication with the ultrasound probe.

At 804, method 800 includes determining a target axial pressure profile of a non-diffracting acoustic beam to be transmitted by the ultrasound probe. The target axial pressure profile may be obtained from the scan protocol or via user input. When imaging in shear-wave elastography imaging mode, the target axial pressure profile may be a square wave function defined by a beam length and a beam center. However, other acquisition modes may utilize other axial pressure profiles and any desired axial pressure profile may be determined. Example target axial pressure profiles are shown in FIG. 17. Therein, a first target axial pressure profile 1702 and a second target axial pressure profile 1704 are shown. Each target axial pressure profile is depicted as normalized pressure along the lateral and axial dimensions. In some examples, the first target axial pressure profile 1702 may be used to generate a non-diffracting acoustic beam for shear wave elastography (SWE) imaging (e.g., a push beam to generate shear waves) while the second target axial pressure profile 1704 may be used to generate a non-diffracting acoustic beam for B-mode imaging. For SWE, the target axial pressure profile may have a moderate beam length in order to provide a sufficient amplitude for the shear wave. For B-mode, generally the target axial pressure profile may have a longer beam length and lower side lobes, as shown in FIG. 17.

At 806, an apodization function and a delay function are determined based on the target axial pressure profile and the transducer configuration. Additional details regarding determining the apodization function and the delay function are presented below with respect to FIG. 9. Briefly, the non-diffracting acoustic beam may be comprised of a plurality of superposed non-diffracting acoustic beam components each having a different wavenumber. A complex function is determined for each non-diffracting acoustic beam, which are weighted, interpolated to the locations of the transducer elements of the transducer, and summed. The distributions of the amplitude and phase for the transducer elements are determined from the complex function for each transducer element, which are used to determine the apodization and delay functions.

At 808, the transducer is controlled based on the apodization and delay functions in order to transmit the non-diffracting acoustic beam. In some examples, to control the transducer based on the apodization and delay functions, a respective electrical signal may be sent to each transducer element of the transducer, and each respective electrical signal may be delayed by a respective delay value based on the delay function and has an amplitude (e.g., voltage) defined by a respective apodization value based on the transmit delay function. In other examples, the apodization function may be encoded into the delay function to form an encoded delay function (which is explained in more detail below with respect to FIG. 9). In such examples, to control the transducer, a respective electrical signal may be sent to each transducer element of the transducer, and each respective electrical signal may be delayed by a respective delay value based on the encoded delay function (and each electrical signal may have the same voltage).

At 810, echoes are received at the transducer. As explained above with respect to FIG. 1, the non-diffracting acoustic beam is back-scattered from structures within the imaging subject/ROI to produce echoes that return to the transducer elements of the ultrasound probe; if the non-diffracting beam is used to generate a shear wave, the echoes that are received may be generated by the propagating shear wave. The echoes are converted into electrical signals by the transducer elements and the electrical signals are processed (e.g., amplified, digitized, delayed, summed) and stored in memory.

At 812, the transmission of the non-diffracting acoustic beam and echo reception may be repeated one or more times as dictated by the acquisition mode and/or scan protocol. In some examples, as indicated at 814, the beam axis of the non-diffracting acoustic beams may be steered in order to transmit the non-diffracting acoustic beams across the ROI to be imaged and obtain sufficient ultrasound data to generate an image (which in some examples may be an elastogram). To steer the beam axis, the electrical signals sent to the transducer elements to produce a given non-diffracting acoustic beam may be delayed (e.g., in addition to the delays for forming the non-diffracting acoustic beam with the target axial pressure profile) to steer the wavefront at a desired angle. In some examples, during shear-wave elastography imaging, after transmission of the non-diffracting acoustic push beam, tracking of the shear wave may be performed using, for example, a plane-wave compounding method (e.g., three angles: −3°, 0°, and 3°).

At 816, an image is generated from the ultrasound data generated from the received echoes. The image may be a B-mode image, Doppler image, a 2D or 3D elastography image (e.g., an elastogram), or another image depending on the acquisition mode. The B-mode image may be a 2D B-mode image, a 3D B-mode image, or a 4D B-mode image (e.g., a cine loop/video). Other example images that may be generated from echoes received from transmitted non-diffracting acoustic beams include color flow images and contrast agent enhanced images. For shear-wave elastography, the image may be an elastogram, which is a map of the velocity of the shear waves in the ROI, where velocity is indicated by color. At 818, the image is saved in memory and/or displayed on a display device. When the image is an elastogram, the image may be displayed over or alongside a B-mode image of the ROI.

It is to be appreciated that a delay function and an apodization function are not necessarily generated each time a non-diffracting acoustic beam is transmitted. Rather, apodization and delay functions (or alternatively, encoded delay functions) for a plurality of different target axial pressure profiles may be predetermined (according to the method of FIG. 9, described below) and stored in memory. Then, during scanning, an apodization function and a delay function (or an encoded delay function) may be retrieved from memory based on the target axial pressure profile dictated by the scan protocol and the configuration of the transducer being used for imaging and used to generate the non-diffracting acoustic beams. When attenuation compensation is applied, the retrieved apodization function and delay function (or retrieved encoded delay function) may be modified based on the attenuation of the material, as described below, or various attenuation-compensated apodization and delay functions (or alternatively, encoded delay functions) may be predetermined for common attenuation coefficients and stored in memory.

FIG. 9 illustrates a method 900 for determining an apodization function and a delay function for generating a non-diffracting beam. Method 900 is described with regard to the systems and components of FIG. 1, though it should be appreciated that the method 900 may be implemented with other systems and components without departing from the scope of the present disclosure. Method 900 may be carried out according to instructions stored in non-transitory memory of a computing device, such as memory 120, and executed by a processor of the computing device, such as processor 116. In some examples, method 900 may be carried out as part of method 800, for example at 806 of method 800. As such, method 900 may determine the apodization function and the delay function based on a target axial pressure profile for the non-diffracting acoustic beam and based on the configuration of the transducer, as explained above with respect to FIG. 8.

At 902, a total number of non-diffracting acoustic beam components is calculated. As explained previously, the non-diffracting acoustic beam may be comprised of a plurality of superposed non-diffracting acoustic beam components. To determine how many non-diffracting acoustic beam components are to be superposed to form the non-diffracting acoustic beam, the total number of non-diffracting acoustic beam components is determined, which may be based on the target axial pressure profile for the non-diffracting beam to be transmitted and based on the frequency of the transducer.

When generating a non-diffracting acoustic beam for shear wave elastography, the target axial pressure profile may be a square wave with a length (referred to as the beam length BL) and a center (referred to as the beam center BC). The axial pressure profile f(z) may be a function of an axial grid z that is determined according to the equation (using 0.1 mm as an axial resolution, though other resolutions are possible):

$$z = 0 : 0.1 mm : BC + \frac{BL}{2} \qquad ( Eq . 1 )$$

Thus, the axial grid z may be defined in increments of 0.1 mm from 0 to a maximum axial depth L of the non-diffracting acoustic beam, which is based on the beam length and the beam center, such that the maximum axial depth L may be calculated according to the following equation:

$$L = BC + \frac{BL}{2} \qquad ( Eq . 2 )$$

In some examples, for

$$BC - \frac{BL}{2} \leq z \leq BC + \frac{BL}{2} ,$$

$$f ( z ) = 1.$$

As explained above, the axial pressure profile may be a square wave (with the same pressure for the entire beam length), but other pressure profiles are possible without departing from the scope of this disclosure.

The total number of non-diffracting acoustic beams (bn) may be determined based on the following equation:

$$bn = \frac{L π f}{2 c} \qquad ( Eq . 3 )$$

Thus, the total number of non-diffracting acoustic beams (bn) is a function of the maximum axial depth L of the non-diffracting acoustic beam, the frequency (f) of the transducer, and the speed of sound (c). The speed of sound may be estimated based on the target tissue/material that is to be imaged, or the speed of sound may be a default speed of sound, such as 1540 m/s which is commonly used in ultrasound imaging. The frequency may be the selected frequency that the probe will operate in when transmitting the non-diffracting acoustic beam. In this way, the total number of non-diffracting acoustic beams may increase for non-diffracting acoustic beams of increasing maximum axial depth. As an example, referring back to FIG. 4, the short beam 402 may have a smaller maximum axial depth than the maximum axial depth of the long beam 404. When the axial pressure profile of the short beam 402 is expanded to multiple non-diffracting acoustic beam components, the total number of non-diffracting acoustic beam components may be smaller than the total number of non-diffracting acoustic beam components calculated when axial pressure profile of the long beam 404 is expanded to multiple non-diffracting acoustic beam components (assuming the same transducer and propagation medium).

At 904, a lateral wavenumber, an axial wavenumber, and an element size is calculated for each non-diffracting acoustic beam component. Each axial wavenumber may be calculated based on the frequency of the transducer, the speed of sound of a medium through which the non-diffracting acoustic beam is to propagate, the target axial pressure profile, and the total number of non-diffracting acoustic beam components. In some examples, explained in more detail below, the axial wavenumber may also take into account the attenuation of the medium though which the non-diffracting beam is to propagate. Each lateral wavenumber may be calculated based on the corresponding axial wavenumber (e.g., for a first non-diffracting acoustic beam component, the axial wavenumber may be calculated and the lateral wavenumber for the first non-diffracting acoustic beam component may be determined based on the axial wavenumber for the first non-diffracting acoustic beam component). The calculation of the lateral and axial wavenumbers is based on the wavenumber k for the non-diffracting acoustic beam, which may be determined based on the frequency f of the transducer and the speed of sound c according to the following equation:

$$k = \frac{2 π f}{c} \qquad ( Eq . 4 )$$

Each axial wavenumber k_{z} may be determined according to the following equation:

$${k}_{z} = \frac{2 π}{( bn - 1 ) L} [ \frac{- ( bn - 1 )}{2} : 1 : \frac{bn - 1}{2} ] \qquad ( Eq . 5 )$$

Thus, a first axial wavenumber may be equal to

$$\frac{2 π}{( bn - 1 ) L} [ \frac{- ( bn - 1 )}{2} ]$$

and each additional axial wavenumber may be calculated by incrementing

$$[ \frac{- ( bn - 1 )}{2} ]$$

up by a value of 1 tor each subsequent axial wavenumber, until

$$[ \frac{bn - 1}{2} ]$$

is reached, for calculation of the final axial wavenumber.

Each lateral wavenumber (kₓ) is determined based on k, the corresponding k_{z} and a baseline of the axial wavenumber (Q), according to the equation:

kₓ=\sqrt{k²−(k_{z}+Q)²} where Q=k−max(k_{=z}) (Eq. 6)

The element size for each non-diffracting acoustic beam component may be determined based on the lateral wavenumber for that non-diffracting acoustic beam component. Each lateral wavenumber corresponds to one element size of the transducer. Said another way, each non-diffracting acoustic beam component may be assigned a respective element size that is based on that non-diffracting acoustic beam component's lateral wavenumber. The element size (dx′) for a non-diffracting beam component is different than (and not necessarily equal to) the element size/dimension for each transducer element (dx). For example, each element size (dx′) may be determined according to the following equation:

$${dx}^{′} = \frac{2.4}{{k}_{x}} \qquad ( Eq . 7 )$$

At 906, method 900 optionally includes compensating for attenuation of the medium through which the non-diffracting acoustic beam is to propagate by calculating complex lateral and axial wavenumbers. The determination of whether or not attenuation is to be compensated may be based on the scan protocol, user input, or another suitable mechanism. If attenuation is to be compensated, the lateral wavenumber may be calculated according to the following equation:

$${k}_{x} = {k}_{x} + \frac{i {k}_{x} α}{k} \qquad ( Eq . 8 )$$

In the above equation 8, a is an attenuation coefficient that may be determined based on the medium in which the non-diffracting acoustic beam is to propagate.

The axial wavenumber, when compensating for attenuation, may be calculated according to the equation:

$${k}_{z} = {k}_{z} + \frac{i ( Q + {k}_{z} ) α}{k} \qquad ( Eq . 9 )$$

At 908, a coefficient of each non-diffracting acoustic beam is calculated. Each coefficient may be a complex weight that will be used during multiplexing of the non-diffracting acoustic beam components and may be determined based the corresponding lateral wavenumber. For example, the coefficient (A (n)) of each non-diffracting acoustic beam component may be calculated according to the equation:

$$A ( n ) = \frac{1}{L} {\int }_{0}^{L} f ( z ) {e}^{i \frac{2 π n}{L} z} dz \qquad ( Eq . 10 )$$

The coefficient (A (n)) of each non-diffracting acoustic beam component may be calculated by performing a Fourier series expansion of the designed axial pressure profile which is used to identify the weights of each non-diffracting acoustic beam component. In the equation 10 above, n represents the index of each non-diffracting acoustic beam component (n∈[1, bn]), f(z) represents the designed axial pressure profile, and L represents the maximum axial depth in calculation. Thus, the complex weight of each non-diffracting acoustic beam component (n∈[1, bn]) may be determined based on the maximum axial depth of the non-diffracting acoustic beam L and the axial pressure distribution f(z) of the non-diffracting acoustic beam. The complex weight is obtained by calculating the Fourier series expansion of the designed axial pressure profile.

At 910, a complex function is calculated for each non-diffracting acoustic beam component (also referred to as a complex beam function). Each complex function may be based on the respective complex weight, the respective lateral wavenumber, and a respective lateral grid for that non-diffracting acoustic beam component (where the lateral grid has a resolution of the element size for that non-diffracting acoustic beam component). The lateral grid may be defined by dx′:x′=min (x):dx′:max (x), where x′ is the lateral grid coordinate with a spatial resolution of dx′. This is the spatial resampling of the transducer lateral coordinate. The lateral grid may thereby divide up the lateral extent of the transducer into increments having the size dx′ and a respective lateral grid may be determined for each non-diffracting acoustic beam component (because each non-diffracting acoustic beam component has a different dx′). In some examples, the complex function pₙ(x′) for each non-diffracting beam component may be calculated according to the following equation:

Pₙ(x′)=A(n)J₀(kₓx′) (Eq. 11)

Thus, the complex function for a given non-diffracting acoustic beam component is calculated based on the coefficient (e.g., complex weight) for the given non-diffracting acoustic beam component and the lateral wavenumber for the given non-diffracting acoustic beam component. In the above equation, J₀(x) is the 0^{th} order Bessel function, which is the commonly used denotation for the 0^{th} order solution of Bessel equation. The 0^{th} order Bessel function may be indicated as:

$${J}_{0} ( {k}_{x} {x}^{′} ) = {\sum }_{m = 0}^{∞} \frac{{( - 1 )}^{m}}{{2}^{2 m} {( m ! )}^{2}} {( {k}_{x} {x}^{′} )}^{2 m} \qquad ( Eq . 12 )$$

The complex functions may thus assign a pressure to each element of the lateral grid, for each non-diffracting acoustic beam component.

At 912, each complex function is interpolated to the element size (e.g., width/lateral dimension) of the ultrasound transducer. As explained above, each non-diffracting acoustic beam component is assigned an element size dx′. The element size dx′ may be larger or smaller than the actual size of each transducer element of the ultrasound transducer. Thus, an interpolation (such as a spline interpolation) may be performed to align the element sizes of the complex functions to the ultrasound transducer element size. The interpolation may produce an interpolated complex function pₙ(x) for each non-diffracting acoustic beam component. Thus, the interpolated complex functions may assign a pressure to each ultrasound transducer element, for each non-diffracting acoustic beam component.

At 914, the interpolated complex functions are summed to form a non-diffracting acoustic beam function (p), which specifies the pressure of each transducer element, according to the equation:

p=sum(pₙ(x)) (Eq. 13)

At 916, method 900 determines if the non-diffracting acoustic beam is to be performed by modulating both apodization and delay of the ultrasound transducer, or if delay-only modulation is to be performed. The determination of whether or not delay-only modulation is to be performed may be based on the scan protocol or user input. Apodization (e.g., pressure modulation) specifies a different voltage pulse for each transducer element. Some types of non-diffracting acoustic beams, such as those used as push beams for shear-wave induction, may demand a higher voltage to produce a higher shear-wave amplitude and thereby induce a better shear-wave signal. However, the acoustic output of diagnostic ultrasound imaging systems is constrained to a maximal output set by regulatory agencies. ADAPT-based pressure apodization may demand considerable voltage differences among the ultrasound transducer elements, e.g., with relatively very high voltages applied to central transducer elements and relatively very low voltages applied to peripheral transducer elements. Keeping the high voltages low enough to prevent exceeding the maximal output or the voltage tolerance of the central transducer elements may cause the low voltages applied to the peripheral transducer elements to generate insufficient pressure for high-quality imaging. Thus, some types of non-diffracting beams may be generated using delay-only modulation, where the amplitude (e.g., voltage) of each electrical signal sent to the transducer elements is the same and only the delay values are modulated among transducer elements.

If delay-only modulation is not to be performed, method 900 proceeds to 918 to determine an apodization function (a), which is the absolute value of the summed interpolated complex functions (e.g., the absolute value of the non-diffracting acoustic beam function p), such that a=abs (p). The apodization function may specify the amplitude of each electrical signal sent to each transducer element. At 920, a delay function (d) is determined, which is the unwrapped phase of the summed interpolated complex functions (e.g., the unwrapped phase of the non-diffracting acoustic beam function p), such that

$$d = \frac{{angle ( p )}_{unwrapped}}{2 π} . \qquad ( Eq . 14 )$$

The delay function may specify the delay value for delaying each electrical signal sent to the transducer elements. Thus, when the ultrasound probe is controlled to transmit the non-diffracting acoustic beam (e.g., at 808 of method 800), a respective electrical signal is sent to each transducer element and each electrical signal has an amplitude (e.g., voltage) defined by the apodization function and is delayed by an amount defined by the delay function.

If it is determined at 916 that delay-only modulation is to performed, method 900 proceeds to 922 to encode the apodization function into the delay function to form an encoded delay function. To encode the apodization function into the delay function, the non-diffracting acoustic beam function p is decomposed into the sum of two-phase functions based on Euler's formula. The non-diffracting acoustic beam function p has a normalized amplitude that can be encoded into a phase value by applying the inverse cosine. The pressure value for a given transducer element defined by the non-diffracting acoustic beam function p is divided into two sub-elements and expressed as phase information. The entire transducer (e.g., all the transducer elements) is divided into two sub-apertures with interleaved elements. Every two consecutive elements include the original complex pressure information (e.g., from the non-diffracting acoustic beam function p) while the pressure amplitude value is the same. The final acoustic field is formed by the superposition of the fields generated by both sub-apertures. With this encoding method, the ADAPT-based beam can be produced with uniform apodization values and an interleaved sawtooth-like phase function. The phase-only method can be used on any linear-array transducer to eliminate concerns about element-voltage limits.

Thus, encoding the apodization function into the delay function may include assigning a first encoded delay function to a first sub-aperture of the transducer, as indicated at 924. The first sub-aperture may comprise the odd-numbered transducer elements (e.g., transducer element 1, transducer element 3, etc.) and does not include any even-numbered transducer elements. The first encoded delay function for the first sub-aperture may be defined according to the equation:

$${d}_{odd} = \frac{{( angle ( p ) - a cos ( abs ( p ) ) )}_{unwrapped}}{2 π} \qquad ( Eq . 15 )$$

Encoding the apodization function into the delay function may further include assigning a second encoded delay function to a second sub-aperture of the transducer, as indicated at 926. The second sub-aperture may comprise the even-numbered transducer elements (e.g., transducer element 2, transducer element 4, etc.) and does not include any odd-numbered transducer elements. The second encoded delay function for the second sub-aperture may be defined according to the equation:

$${d}_{even} = \frac{{( angle ( p ) + a cos ( abs ( p ) ) )}_{unwrapped}}{2 π} \qquad ( Eq . 16 )$$

In this way, the delay function may be based on an inverse cosine of the absolute value of the summed interpolated complex beam functions plus or minus an unwrapped phase of the summed interpolated complex beam functions. Thus, when the ultrasound probe is controlled to transmit the non-diffracting acoustic beam (e.g., at 808 of method 800) and delay-only modulation is performed, a respective electrical signal is sent to each transducer element and each electrical signal has the same amplitude (e.g., voltage) and is delayed by an amount defined by the first encoded delay function or the second encoded delay function.

FIG. 10 shows the effect of tissue attenuation and compensation on the acoustic pressure of a non-diffracting acoustic beam. When an acoustic wave propagates in a medium, it undergoes attenuation caused by scattering and absorption along the propagation path. Although ADAPT-based non-diffracting acoustic beams as described herein retain the non-diffraction feature of the Bessel beam, attenuation can alter the desired beam profile and potentially “defocus” the acoustic beam. In conventional, focused-push-beam shear-wave imaging, attenuation shortens the effective region of shear-wave generation and reduces the signal-to-noise ratio (SNR) of the shear wave in deeper regions. Furthermore, the shear-wave amplitude becomes non-uniform in the area of interest, which causes imaging errors. Therefore, attenuation may be compensated using ADAPT to sustain the desired beam profile at greater depth. ADAPT-based attenuation compensation first considers a lossy Helmholtz equation for an acoustic wave: ∇²p+k′²p=0, where the wavenumber k′ is a complex number containing attenuation: k′=k+ia. Thus, a damped plane-wave solution of the Helmholtz equation can be obtained as: p=p₀e^{-az}e^{i(kz-wt)}, where the attenuation coefficient α is embedded. Because the ADAPT-based beam can be considered to be the interference of multiple plane waves (angular spectrum representation), the ADAPT beam-shaping process can be performed with a complex k′ to compensate for attenuation in the medium. As explained above with respect to FIG. 9, for a single Bessel beam: p(x,z)=J₀ (kₓx) e^{ikzz}, longitudinal and transverse wavenumbers are substituted in the complex vector for the attenuation. Then, every Bessel beam and corresponding weight are calculated and multiplexed to form an attenuation-compensated non-diffracting acoustic beam.

FIG. 10 depicts three numerical-simulation scenarios to illustrate the effect of attenuation compensation. A first acoustic field 1002 and corresponding first plot 1004 of an axial center line pressure distribution depicts an example where a first non-diffracting acoustic beam propagates in a medium with no attenuation and no compensation is performed; the attenuation-free non-diffracting beam has a high-intensity region centered at 20 mm. A second acoustic field 1006 and corresponding second plot 1008 of an axial center line pressure distribution shows an example of a second non-diffracting beam (with the same profile of the first non-diffracting acoustic beam) propagating in a medium with attenuation (but without compensation) such that, when attenuation is included, the peak intensity moves ˜2.5 mm closer to the transducer. As shown by a third acoustic field 1010 and corresponding third plot 1012 of an axial center line pressure distribution, when a third non-diffracting acoustic beam (with the same profile as the first and second beams) propagates in the medium with the attenuation and compensation for the attenuation is applied, the third non-diffracting acoustic beam retains the original axial location of the peak intensity, and the energy is more evenly distributed about the beam center.

FIG. 11 schematically depicts a process 1100 for performing delay-only modulation. As explained above with respect to FIG. 9, performing delay-only modulation includes dividing the ultrasound transducer (shown schematically as acoustic transducer 1102 in FIG. 11) into two sets of interleaved elements and applying a respective encoded delay function to each set of elements. Two consecutive elements form an overall element 1104 that is divided into a first sub-element 1106 and second sub-element 1108, with the first encoded delay function (p₁) used to assign a delay value to the first sub-element 1106 and the second encoded delay function (p₂) used to assign a delay value to the second sub-element 1108, and where each of the first sub-element 1106 and the second sub-element 1108 have the same pressure/amplitude.

A schematic 1110 of the element dividing for delay-only modulation is also shown in FIG. 11, with corresponding acoustic pressure of each part of the aperture. The entire aperture is divided into two sub-apertures with interleaved elements, including a first sub-aperture 1112 and a second sub-aperture 1114. Each sub-aperture can form part of the non-diffracting acoustic beam, and then the sub-apertures are superposed to shape the final non-diffracting acoustic beam. It is to be appreciated that an active element in the first sub-aperture 1112 is a non-active element in the second sub-aperture 1114 and vice versa. A simulated acoustic field 1116 of a non-diffracting acoustic beam obtained through delay-only modulation is shown, including a comparison of apodization (shown in plot 1118) and delay (shown in plot 1120) before and after the delay modulation. The information of apodization is encoded into the delay function with a sawtooth distribution.

FIG. 12 illustrates results of delay-only modulation when used in shear-wave imaging, which includes elastograms generated with non-diffracting acoustic beams relative to elastograms generated with traditional focused beams. As shown in FIG. 12 and explained below, the non-diffracting acoustic beam produces a better contrast-to-noise ratio (CNR) and more accurate average shear-wave speed (SWS) estimate than the focused beam. To generate the elastograms, a cylindrical inclusion was embedded in a phantom, where the inclusion has different stiffness values than the surrounding medium of the phantom. The distance between the phantom and transducer was varied to image the inclusion at different depths, as shown schematically at 1202. Two distances between the transducer and the phantom inclusion were used (15 mm and 20 mm); the corresponding non-diffracting acoustic beam (as a push beam) was used for both distances (e.g., with a beam center equaling the inclusion depth, with a beam length of 20 mm). In addition, two different focused-beam focal-zone centers were configured and compared with the non-diffracting acoustic beam with the same beam centers. Inclusion delineation performance is compared between the focused beam and the non-diffracting acoustic beam, as shown by the elastograms 1204 (scale bar: 2 mm). The focused-beam shear-wave image depicts a more-elliptical shape for the cylindrical inclusion, while only one position is well-delineated for the focused-beam. Delineation of the inclusion is incomplete when the focused-beam center is at a different depth than the inclusion center. In comparison, delineation of the inclusion using the non-diffraction acoustic push beam for both relative positions and both beam centers produces a circular shape that is consistent with the circular cross sections of the cylindrical inclusions. The CNR and SWS inside the inclusion were calculated and compared between the cases using the focused beam and the non-diffracting acoustic beam, shown by plots 1206. The non-diffracting acoustic beam based shear waves provide a better CNR and an inclusion SWS estimate that is closer to the calibrated value for the inclusions at both inclusion depths. These results indicate that non-diffracting acoustic beams generated using ADAPT can substantially improve ARFI shear-wave imaging, and the induced quasi-cylindrical shear wave it generates can better delineate constituent components that have different stiffness values than their surroundings.

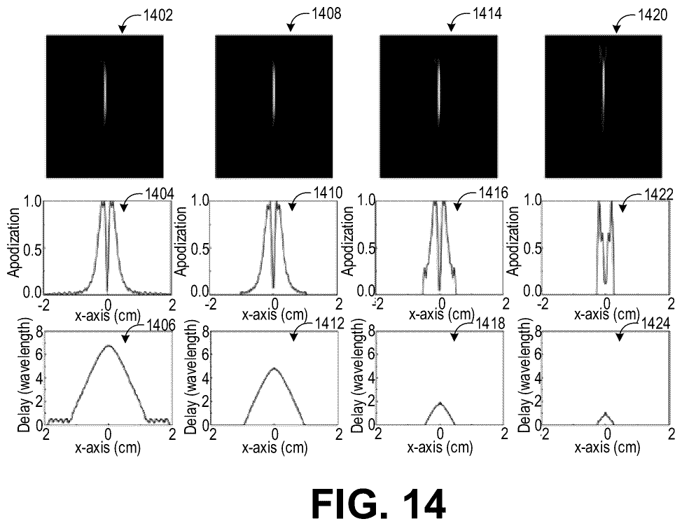

The non-diffracting acoustic beams described herein can be generated with a variety of different transducer configurations. As one example, a non-diffracting acoustic beam with a selected axial pressure profile may be generated with different numbers of transducer elements used to form the non-diffracting acoustic beam. FIG. 14 shows four example non-diffracting acoustic beams all having the same axial pressure profile and produced with different transducer elements. A first non-diffracting acoustic beam 1402, shown as an acoustic field, may be formed by applying a first apodization function 1404 and a first delay function 1406. The first apodization function 1404 and the first delay function 1406 may be determined based on a target axial pressure profile for the first non-diffracting acoustic beam 1402 and a configuration of the transducer, which in this example includes a first number of transducer elements (e.g., 192 elements).

A second non-diffracting acoustic beam 1408, shown as an acoustic field, may be formed by applying a second apodization function 1410 and a second delay function 1412. The second apodization function 1410 and the second delay function 1412 may be determined based on the target axial pressure profile for the second non-diffracting acoustic beam 1408 (which is the same as the target axial pressure profile for the first non-diffracting acoustic beam) and a configuration of the transducer, which in this example includes a second number of transducer elements (e.g., 96 elements).

A third non-diffracting acoustic beam 1414, shown as an acoustic field, may be formed by applying a third apodization function 1416 and a third delay function 1418. The third apodization function 1416 and the third delay function 1418 may be determined based on the target axial pressure profile for the third non-diffracting acoustic beam 1414 (which is the same as the target axial pressure profile for the first non-diffracting acoustic beam) and a configuration of the transducer, which in this example includes a third number of transducer elements (e.g., 48 elements).

A fourth non-diffracting acoustic beam 1420 shown as an acoustic field, may be formed by applying a fourth apodization function 1422 and a fourth delay function 1424. The fourth apodization function 1422 and the fourth delay function 1424 may be determined based on the target axial pressure profile for the fourth non-diffracting acoustic beam 1420 (which is the same as the target axial pressure profile for the first non-diffracting acoustic beam) and a configuration of the transducer, which in this example includes a fourth number of transducer elements (e.g., 24 elements).

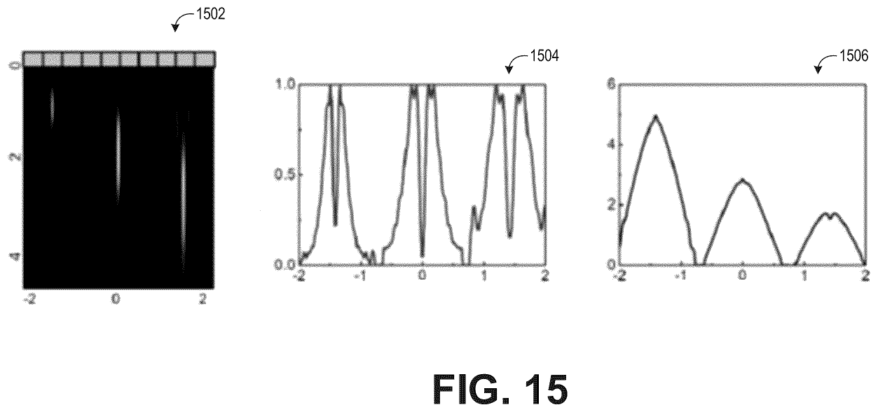

FIG. 15 shows an example of multiple non-diffracting acoustic beams generated simultaneously with the same transducer. Specifically, FIG. 15 shows the acoustic fields 1502 for the three non-diffracting acoustic beams, an apodization function 1504 for generating the three non-diffracting acoustic beams, and a delay function 1506 for generating the three non-diffracting acoustic beams. Thus, multiple non-diffracting acoustic beams of various beam depths and beam lengths may be generated at the same time.

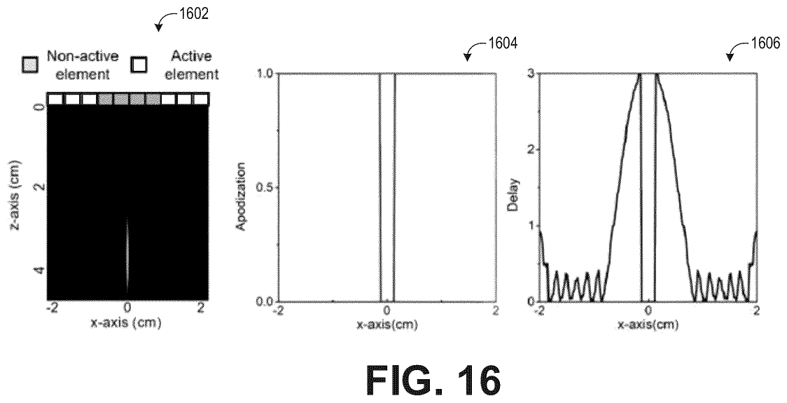

FIG. 16 shows an example non-diffracting acoustic beam generated with a split aperture. Specifically, FIG. 16 shows the acoustic field 1602 for the non-diffracting acoustic beam, an apodization function 1604 for generating the non-diffracting acoustic beam, and a delay function 1606 for generating the non-diffracting acoustic beam. The split aperture may be formed by designating transducer elements in the center of the transducer as non-active elements.

Thus, a transmit beamshaping method is disclosed herein, termed ADAPT, for generating propagation-invariant acoustic beams (referred to as non-diffracting acoustic beams). ADAPT-based beams can be shaped with arbitrary longitudinal pressure distributions over extended axial distances. ADAPT-based beams can be generated with a single, linear-array transducer utilizing Bessel-beam multiplexing. This beam-generation method offers a high degree of freedom for controlling the longitudinal acoustic energy. The ADAPT method may be optimized for a variety of applications. The non-diffracting nature of ADAPT-based beams permits compensation for acoustic attenuation and diffraction in the insonified material by incorporating the attenuation coefficient in the beam-shaping process. As a result, the acoustic beam effectively can maintain the desired beam profile during propagation. Finally, the phase/delay-only version of ADAPT allows uniform pressure/apodization and enables the ultrasound system to remain below maximum voltage and output-pressure limitations. ADAPT can be used in acoustic systems where pressure modulation is unavailable, and it can substantially reduce the complexity of beam shaping.

ADAPT beamshaping may be applied to ARFI shear-wave imaging, which has great potential clinical value, e.g., for disease diagnosis, therapy monitoring, and treatment planning. ARFI shear-wave imaging displays the tissue shear-modulus distribution, can follow tissue stiffness changes over time, and can map the location and shape of abnormal regions. Most current shear-wave imaging uses the classic focused-beam method to generate push beams for ARFI shear-wave elastography. Although it can focus acoustic energy, the highly localized shear-wave push beam can produce errors in a wide imaging area and difficulties in producing an overview of the stiffness of the tissue. The standard non-diffracting Bessel beam has a fixed beam profile starting from the surface of the acoustic source. Thus, its capability for deep imaging is limited. In comparison, the ADAPT method provides a versatile tool for shear-wave generation at desired locations and can fit with various dimensions of the imaging target/area. Furthermore, the ADAPT framework can be applied to different types of transducers (e.g., linear, curved, and 2D arrays). ARFI shear-wave imaging using ADAPT-based push beams can provide accurate inclusion delineation. Combining all features of the ADAPT (e.g., beam shaping and attenuation compensation) can provide a general format for diagnostic and therapeutic ultrasound and takes a substantial step toward applying optimal acoustic beams for a wide variety of applications in the complicated clinical environment.

A technical effect of beamshaping ultrasound transmit beams using ADAPT to form non-diffracting acoustic beams is that non-diffracting/propagation-invariant beams may be generated with any desired axial pressure profile using standard ultrasound transducer configurations (e.g., linear array, curved array, etc.). Another technical effect is that non-diffracting acoustic beams shaped according to ADAPT may generate shear waves at deeper depths than standard focused beams, thereby improving shear-wave imaging and the delineation of constituent components that have different stiffness values than their surroundings.

The disclosure also provides support for a method for transmitting a non-diffracting acoustic beam with an ultrasound transducer that includes a plurality of transducer elements, the method comprising: determining a transmit delay function and a transmit apodization function for the ultrasound transducer based on a target axial pressure profile for the acoustic beam for a given configuration of the ultrasound transducer, and controlling the ultrasound transducer to transmit the acoustic beam by sending electrical signals to the plurality of transducer elements based on the transmit delay function and the transmit apodization function. In a first example of the method, the target axial pressure profile for the acoustic beam is an arbitrary function defined by a user. In a second example of the method, optionally including the first example, the target axial pressure profile for the acoustic beam is defined as a square wave function with a length and center that are each selected based on a region of interest (ROI) to be imaged via the acoustic beam. In a third example of the method, optionally including one or both of the first and second examples, the method further comprises: receiving, at the ultrasound transducer, echoes from the ROI following transmission of the acoustic beam, generating two-dimensional (2D) or three-dimensional (3D) elastography information from the received echoes, and displaying the 2D or 3D elastography information on a display device. In a fourth example of the method, optionally including one or more or each of the first through third examples, the method further comprises: receiving, at the ultrasound transducer, echoes from a region of interest (ROI) following transmission of the acoustic beam to the ROI, generating a 2D, 3D, or 4D B-mode image, a Doppler image, a color flow image, or a contrast agent enhanced image from the received echoes, and displaying the 2D, 3D, or 4D B-mode image, the Doppler image, the color flow image, or the contrast agent enhanced image on a display device. In a fifth example of the method, optionally including one or more or each of the first through fourth examples, the configuration of the ultrasound transducer includes a frequency of the ultrasound transducer, a number of transducer elements comprising the plurality of transducer elements, a dimension of each transducer element, and a coordinate of each transducer element along an axis of the ultrasound transducer. In a sixth example of the method, optionally including one or more or each of the first through fifth examples, the transmit delay function and the transmit apodization function are further determined based on a speed of sound and/or an attenuation of a material through which the acoustic beam is to propagate. In a seventh example of the method, optionally including one or more or each of the first through sixth examples, a respective electrical signal is sent to each transducer element, and each respective electrical signal is delayed by a respective delay value based on the transmit delay function and has an amplitude defined by a respective apodization value based on the transmit apodization function. In a eighth example of the method, optionally including one or more or each of the first through seventh examples, controlling the ultrasound transducer to transmit the acoustic beam by sending electrical signals to the plurality of transducer elements based on the transmit delay function and the transmit apodization function comprises encoding the transmit apodization function into the transmit delay function to form an encoded delay function, and controlling the ultrasound transducer to transmit the acoustic beam by sending a respective electrical signal to each transducer element that is delayed by a respective delay value based on the encoded delay function.

The disclosure also provides support for a method for transmitting a non-diffracting acoustic beam with a transducer of an ultrasound probe, comprising: calculating a total number of non-diffracting acoustic beam components based on a target axial pressure profile for the non-diffracting acoustic beam to be transmitted and a frequency of the transducer, calculating, for each non-diffracting acoustic beam component, a respective lateral wavenumber, a respective axial wavenumber, a respective complex weight, and a respective element size, interpolating each non-diffracting acoustic beam component to a transducer element size of the transducer based on the respective element size of each non-diffracting acoustic beam component, summing the interpolated non-diffracting acoustic beam components, determining an apodization function and a delay function for the non-diffracting acoustic beam based on the summed interpolated non-diffracting acoustic beam components, and controlling the transducer to transmit the non-diffracting acoustic beam by activating a plurality of elements of the transducer according to the apodization function and the delay function. In a first example of the method, the target axial pressure profile comprises a target beam length for the non-diffracting acoustic beam and a target beam center for the non-diffracting acoustic beam. In a second example of the method, optionally including the first example, calculating, for each non-diffracting acoustic beam component, the respective lateral wavenumber, the respective axial wavenumber, the respective complex weight, and the respective element size comprises: calculating, for each non-diffracting acoustic beam component, the respective axial wavenumber based on the frequency of the transducer, a speed of sound and an attenuation of a medium through which the non-diffracting acoustic beam is to propagate, the target axial pressure profile, and the total number of non-diffracting acoustic beam components, determining, for each non-diffracting acoustic beam component, the respective lateral wavenumber based on the respective axial wavenumber, determining, for each non-diffracting acoustic beam component, the respective element size based on the respective lateral wavenumber, assigning, to each non-diffracting acoustic beam component, the respective complex weight based on the respective lateral wavenumber, and calculating, for each non-diffracting acoustic beam component, a respective complex beam function based on the respective complex weight, the respective lateral wavenumber, and a lateral grid having a resolution of the respective element size. In a third example of the method, optionally including one or both of the first and second examples, interpolating each non-diffracting acoustic beam component to the transducer element size of the transducer comprises performing a spline interpolation of each respective complex beam function to form respective interpolated complex beam functions and wherein summing the interpolated non-diffracting acoustic beam components comprises summing the respective interpolated complex beam functions. In a fourth example of the method, optionally including one or more or each of the first through third examples, the apodization function is the absolute value of the summed interpolated complex beam functions and the delay function is an unwrapped phase of the summed interpolated complex beam functions. In a fifth example of the method, optionally including one or more or each of the first through fourth examples, the apodization function is a square wave function and the delay function is calculated based on an inverse cosine of the absolute value of the summed interpolated complex beam functions plus or minus an unwrapped phase of the summed interpolated complex beam functions.

The disclosure also provides support for a system, comprising: an ultrasound probe including a transducer comprising a plurality of elements, a display device, memory storing instructions, and one or more processors configured to execute the instructions to: control the ultrasound probe to generate a shear wave at a region of interest (ROI) in a patient by transmitting a non-diffracting acoustic beam having a target axial pressure profile, the non-diffracting acoustic beam comprised of a plurality of superposed non-diffracting acoustic beam components, receive, with the ultrasound probe, echoes from the ROI, generate elastography information from the received echoes, and display the elastography information on the display device. In a first example of the system, the target axial pressure profile comprises a target beam length for the non-diffracting acoustic beam and a target beam center for the non-diffracting acoustic beam, and wherein the target beam length and the target beam center are selected based on the ROI. In a second example of the system, optionally including the first example, controlling the ultrasound probe to transmit the non-diffracting acoustic beam comprises controlling each element of the plurality of elements according to an apodization function and a delay function each calculated based on the superposed non-diffracting acoustic beam components, the superposed non-diffracting acoustic beam components determined based on the target axial pressure profile, a configuration of the transducer, and a speed of sound of a medium in which the non-diffracting acoustic beam is to propagate. In a third example of the system, optionally including one or both of the first and second examples, the apodization function and the delay function are further based on an attenuation of the medium. In a fourth example of the system, optionally including one or more or each of the first through third examples, the plurality of elements of the ultrasound probe are arranged into a linear array, a curved array, or a two-dimensional array, and wherein the one or more processors are further configured to execute the instructions to: control the ultrasound probe to simultaneously transmit multiple non-diffracting acoustic beams distributed laterally with respect to the plurality of elements, and/or control the ultrasound probe to transmit non-diffracting acoustic beams with a beam axis that is steered at an angle relative to the plurality of elements.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

The invention claimed is:

1. A method for transmitting a non-diffracting acoustic beam with an ultrasound transducer that includes a plurality of transducer elements, the method comprising:
determining a transmit delay function and a transmit apodization function for the ultrasound transducer based on a target axial pressure profile for the acoustic beam for a given configuration of the ultrasound transducer; and
controlling the ultrasound transducer to transmit the acoustic beam by sending electrical signals to the plurality of transducer elements based on the transmit delay function and the transmit apodization function.

2. The method of claim 1, wherein the target axial pressure profile for the acoustic beam is an arbitrary function defined by a user.

3. The method of claim 1, wherein the target axial pressure profile for the acoustic beam is defined as a square wave function with a length and center that are each selected based on a region of interest (ROI) to be imaged via the acoustic beam.

4. The method of claim 3, further comprising:
receiving, at the ultrasound transducer, echoes from the ROI following transmission of the acoustic beam;
generating two-dimensional (2D) or three-dimensional (3D) elastography information from the received echoes; and
displaying the 2D or 3D elastography information on a display device.

5. The method of claim 1, further comprising:
receiving, at the ultrasound transducer, echoes from a region of interest (ROI) following transmission of the acoustic beam to the ROI;
generating a 2D, 3D, or 4D B-mode image, a Doppler image, a color flow image, or a contrast agent enhanced image from the received echoes; and
displaying the 2D, 3D, or 4D B-mode image, the Doppler image, the color flow image, or the contrast agent enhanced image on a display device.

6. The method of claim 1, wherein the configuration of the ultrasound transducer includes a frequency of the ultrasound transducer, a number of transducer elements comprising the plurality of transducer elements, a dimension of each transducer element, and a coordinate of each transducer element along an axis of the ultrasound transducer.

7. The method of claim 1, wherein the transmit delay function and the transmit apodization function are further determined based on a speed of sound and/or an attenuation of a material through which the acoustic beam is to propagate.

8. The method of claim 1, wherein a respective electrical signal is sent to each transducer element, and each respective electrical signal is delayed by a respective delay value based on the transmit delay function and has an amplitude defined by a respective apodization value based on the transmit apodization function.

9. The method of claim 8, wherein controlling the ultrasound transducer to transmit the acoustic beam by sending electrical signals to the plurality of transducer elements based on the transmit delay function and the transmit apodization function comprises encoding the transmit apodization function into the transmit delay function to form an encoded delay function, and controlling the ultrasound transducer to transmit the acoustic beam by sending a respective electrical signal to each transducer element that is delayed by a respective delay value based on the encoded delay function.

10. A method for transmitting a non-diffracting acoustic beam with a transducer of an ultrasound probe, comprising:
calculating a total number of non-diffracting acoustic beam components based on a target axial pressure profile for the non-diffracting acoustic beam to be transmitted and a frequency of the transducer;
calculating, for each non-diffracting acoustic beam component, a respective lateral wavenumber, a respective axial wavenumber, a respective complex weight, and a respective element size;
interpolating each non-diffracting acoustic beam component to a transducer element size of the transducer based on the respective element size of each non-diffracting acoustic beam component;
summing the interpolated non-diffracting acoustic beam components;
determining an apodization function and a delay function for the non-diffracting acoustic beam based on the summed interpolated non-diffracting acoustic beam components; and
controlling the transducer to transmit the non-diffracting acoustic beam by activating a plurality of elements of the transducer according to the apodization function and the delay function.

11. The method of claim 10, wherein the target axial pressure profile comprises a target beam length for the non-diffracting acoustic beam and a target beam center for the non-diffracting acoustic beam.

12. The method of claim 10, wherein calculating, for each non-diffracting acoustic beam component, the respective lateral wavenumber, the respective axial wavenumber, the respective complex weight, and the respective element size comprises:
calculating, for each non-diffracting acoustic beam component, the respective axial wavenumber based on the frequency of the transducer, a speed of sound and an attenuation of a medium through which the non-diffracting acoustic beam is to propagate, the target axial pressure profile, and the total number of non-diffracting acoustic beam components;
determining, for each non-diffracting acoustic beam component, the respective lateral wavenumber based on the respective axial wavenumber;
determining, for each non-diffracting acoustic beam component, the respective element size based on the respective lateral wavenumber;
assigning, to each non-diffracting acoustic beam component, the respective complex weight based on the respective lateral wavenumber; and
calculating, for each non-diffracting acoustic beam component, a respective complex beam function based on the respective complex weight, the respective lateral wavenumber, and a lateral grid having a resolution of the respective element size.

13. The method of claim 12, wherein interpolating each non-diffracting acoustic beam component to the transducer element size of the transducer comprises performing a spline interpolation of each respective complex beam function to form respective interpolated complex beam functions and wherein summing the interpolated non-diffracting acoustic beam components comprises summing the respective interpolated complex beam functions.

14. The method of claim 13, wherein the apodization function is the absolute value of the summed interpolated complex beam functions and the delay function is an unwrapped phase of the summed interpolated complex beam functions.

15. The method of claim 13, wherein the apodization function is a square wave function and the delay function is calculated based on an inverse cosine of the absolute value of the summed interpolated complex beam functions plus or minus an unwrapped phase of the summed interpolated complex beam functions.

16. A system, comprising:
an ultrasound probe including a transducer comprising a plurality of elements;
a display device;
memory storing instructions; and
one or more processors configured to execute the instructions to:
control the ultrasound probe to generate a shear wave at a region of interest (ROI) in a patient by transmitting a non-diffracting acoustic beam having a target axial pressure profile, the non-diffracting acoustic beam comprised of a plurality of superposed non-diffracting acoustic beam components;
receive, with the ultrasound probe, echoes from the ROI;
generate elastography information from the received echoes; and
display the elastography information on the display device.

17. The system of claim 16, wherein the target axial pressure profile comprises a target beam length for the non-diffracting acoustic beam and a target beam center for the non-diffracting acoustic beam, and wherein the target beam length and the target beam center are selected based on the ROI.

18. The system of claim 17, wherein controlling the ultrasound probe to transmit the non-diffracting acoustic beam comprises controlling each element of the plurality of elements according to an apodization function and a delay function each calculated based on the superposed non-diffracting acoustic beam components, the superposed non-diffracting acoustic beam components determined based on the target axial pressure profile, a configuration of the transducer, and a speed of sound of a medium in which the non-diffracting acoustic beam is to propagate.

19. The system of claim 18, wherein the apodization function and the delay function are further based on an attenuation of the medium.

20. The system of claim 16, wherein the plurality of elements of the ultrasound probe are arranged into a linear array, a curved array, or a two-dimensional array, and wherein the one or more processors are further configured to execute the instructions to:
control the ultrasound probe to simultaneously transmit multiple non-diffracting acoustic beams distributed laterally with respect to the plurality of elements; and/or
control the ultrasound probe to transmit non-diffracting acoustic beams with a beam axis that is steered at an angle relative to the plurality of elements.
